(19) United States
(12) Patent Application Publication
(10) Pub. No.: US 2011/0022113 A1
(43) Pub. Date: Jan. 27, 2011

(54) Analyzer Compatible Communication Protocol

(76) Inventors: Mark Zdeblick, Portola Valley, CA (US); Lawrence Arne, Redwood City, CA (US); Nilay Jani, Palo Alto, CA (US); Haifeng Li, Sunnyvale, CA (US); Jonathan Withrington, San Francisco, CA (US); Benedict J. Costello, Berkeley, CA (US); Alexander Gilman, Redwood City, CA (US); Adam Whitworth, Los Altos, CA (US)

Correspondence Address: Proteus Biomedical, Inc.;Bozicevic, Field & Francis LLP, 1900 University Avenue, Suite 200, East Palo Alto, CA, 94303, US

(21) Appl. No.: 12/669,031

(22) PCT Filed: Nov. 30, 2009
(86) PCT No.: PCT/US09/66130

§ 371 Date: Jan. 13, 2010

Related U.S. Application Data
(60) Provisional application No. 61/119,348, filed on Dec. 2, 2008.

(51) Int. Cl. A61N 1/08

(52) U.S. Cl. 607/30

(57) ABSTRACT

Methods and systems for programming a plurality of leads under at least two distinct modalities are provided. The leads may be grouped within satellites and multiple satellites may be configured within a single lead. Each lead includes a power and communications bus providing commands, and information and pulses to the satellites. The leads may be connected to at least two different command and pulse sources, optionally a cardiac pacemaker and/or a cardiac pulse analyzer system. A command may include or be preceded by a wake-up pulse that facilitates identification of a modality applicable to the associated command and data. A command may further optionally include a reference pulse or series of reference pulses, whereby the satellite references data pulses in relation to one or more aspects of the associated reference pulse. A data pulse may deliver two bits of information.

### RELATED APPLICATION AND CROSS REFERENCE

This application claims the benefit of U.S. Provisional Application Ser. No. 61/119,348 filed on Dec. 2, 2008, the entire disclosure of which is incorporated herein by reference.

### FIELD OF THE INVENTION

The present invention relates to administering electromagnetic signals to local areas of living tissue. In particular, the present invention relates to systems and techniques for controlling two or more effectors, e.g., electrodes, which can be used to administer electromagnetic signals to living tissue.

### INTRODUCTION

Electrodes for administering electrical signals for monitoring electrical signals at specific locations in living tissue, such as the heart, are important tools used in many medical treatments or diagnoses. Certain legacy pacemakers employ individual electrodes coupled to a control circuit wherein the control circuit directs pacing pulses through each of a plurality of two wire connections to isolated electrodes. Each two-wire power connection may be dedicated to a single electrode. Related commercially available instrumentation exists, e.g., heart pacing pulse generators. The heart pacing pulse generators may be used, for example, to excite pluralities of individual electrodes, wherein each individual electrode is separately coupled through a dedicated two-wire connection. The heart pacing pulse generators are designed to provide pacing pulses of variable amplitudes and voltages to individual electrodes and to perform impedance measurements.

Various lead configurations are also available as are two-conductor bus systems for connecting physiologic sensors to a pacemaker. The two-conductor bus provides power to the sensors, and the sensors' output signals are modulated on the two wires.

The application of programmable multi-electrode lead systems requires the selection of programming control circuitry or instrumentation that delivers commands in a modality that can be interpreted by a receiving programmable lead electrode system, e.g., a satellite having at least one electrode, as a command. Therefore, the possibility of applying legacy pacing pulse generators for use in directing the performance of a programmable electrode may be limited by the range of electrical signals that the legacy pacing pulse generator can use to provide as programming information.

### SUMMARY OF THE INVENTION

The present invention may address at least some of the foregoing issues, wherein methods and systems for programming a multi-electrode lead system with at least two modalities of command are provided. In certain aspects, a central controller may program the multi-electrode lead system in a first modality and a separate pulse generator may program the same multi-lead system in a second modality.

It is understood that the terms “pulse” and “waveform” are used synonymously in the present disclosure.

The subject methods and systems find use in a variety of different applications, including cardiac resynchronization therapy, kinesiology, monitoring or exciting of organic tissue, neurological examination and therapy, and gastrointestinal examination and therapy.

The foregoing and other objects, features and advantages will be apparent from the following description of aspects of the present invention as illustrated in the accompanying drawings.

### INCORPORATION BY REFERENCE

All publications mentioned herein are incorporated herein by reference to disclose and describe the methods and/or materials in connection with which the publications are cited. All publications, patents, and patent applications mentioned in this specification are herein incorporated by reference in their entirety and for all purposes to the same extent as if each individual publication, patent, or patent application was specifically and individually indicated to be incorporated by reference.

Such incorporations included the U.S. Provisional Patent Application Nos. 60/707,995, filed Aug. 12, 2005; 60/679,625, filed May 9, 2005; 60/638,928, filed Dec. 23, 2004; 60/607,280, filed Sep. 2, 2004; U.S. patent application Ser. Nos. 10/764,127, filed Jan. 23, 2004; 10/764,429, filed Jan. 23, 2004; 10/764,125, filed Jan. 23, 2004; and 10/734,490, filed Dec. 11, 2003.

The publications discussed or mentioned herein are provided solely for their disclosure prior to the filing date of the present application. Nothing herein is to be construed as an admission that the present invention is not entitled to antedate such publication by virtue of prior invention. Furthermore, the dates of publication provided herein may differ from the actual publication dates which may need to be independently confirmed.

### BRIEF DESCRIPTION OF THE FIGURES

These, and further features of various aspects of the present invention, may be better understood with reference to the accompanying specification and drawings depicting various aspects of the present invention, in which:

FIG. 1 is a high level schematic of a cardiac pacing and signal detection system in which a number of satellite units have two or more electrodes.

FIG. 2 is a detailed schematic of an exemplary right ventricular lead of FIG. 1 that includes four satellites.

FIG. 3 is a detailed schematic of a legacy cardiac pacing pulse analyzer coupled with the right ventricular lead of FIGS. 1 and 2.

FIG. 4 is a detailed schematic of the first satellite of the right ventricular lead of FIGS. 1 through 3.

FIG. 5 is a table of symbols used to program an electrode configuration of each satellite of the right ventricular lead of FIGS. 1 through 4.

FIG. 6 is a table of symbols and the commands that the symbols represent as used to an electrode configuration of each satellite of the right ventricular lead of FIGS. 1 through 4.

FIG. 7 is a table of symbols used to program an electrode configuration of each satellite of the right ventricular lead of FIGS. 1 through 4;

FIG. 8 is a timing diagram of a sample command formatted by the cardiac pacing pulse analyzer of FIG. 2.

FIG. 9 is a high frequency wakeup command formatted in accordance with a first modality and as generated by the central controller of FIGS. 1 and 3.

FIG. 10 is an illustration of a structure of commands that may vary in formatting between an electrical signal formatting of the first modality relevant to the central controller of FIG. 1 and an electrical signal formatting of a second modality relevant to the legacy cardiac pacing pulse analyzer of FIG. 3.

FIG. 11 is a table that illustrates command encoding according to an ordering of pulses within a command intended to program, control or manage the satellites of FIGS. 1 through 4.

FIG. 12 is a table of use cases of commands applicable to program a satellite of FIGS. 1 through 4.

FIGS. 13 and 14 are illustrations of additional aspects of the first satellite of FIGS. 1 through 4 useful for extraction of information from electrical signals transmitted from the central controller of FIGS. 1 and 2 and the legacy cardiac pacing pulse analyzer of FIG. 3.

### DETAILED DESCRIPTION

It is to be understood that this invention is not limited to particular aspects of the present invention described, as such may, of course, vary. It is also to be understood that the terminology used herein is for the purpose of describing particular aspects only, and is not intended to be limiting, since the scope of the present invention will be limited only by the appended claims.

Methods recited herein may be carried out in any order of the recited events which is logically possible, as well as the recited order of events.

Where a range of values is provided herein, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the methods and materials are now described.

It must be noted that as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

Aspects of the present invention provide techniques and systems adaptable for use with in evaluating the motion, state or position of an organ or a living tissue of a living being. The living being may be an animal, or more particularly a “mammal” or “mammalian,” where these terms are used broadly to describe organisms which are within the class mammalia, including the orders carnivore, e.g., dogs and cats, rodentia, e.g., mice, guinea pigs, and rats, lagomorpha, e.g. rabbits and primates, e.g., humans, chimpanzees, and monkeys. In many applications, the subjects or patients will be humans.

The first method may be applied to living tissue and/or organs of living beings, such as a heart, a lung, a kidney, a limb, a section of dermis, a hand, a foot, a gut area, a digestive tissue, a bone, cartilage, and/or a muscle. According to the first method, an electromagnetic pulse may be delivered to living tissue at a cardiac location, such as at or proximate to a heart wall or an element of the diaphragm.

In the subject methods, an electrode may be stably associated with a tissue location of a living being, and an application of an energy pulse or an energetic field to a tissue location may be performed by the associated electrode.

“Evaluating” is used herein to refer to any type of detecting, assessing or analyzing, and may be qualitative or quantitative. The tissue location evaluated in accordance with the various aspects is generally a defined location or portion of a body, i.e., subject, where in many cases it is a defined location or portion, i.e., domain or region, of a body structure, such as an organ, where in representative applications the body structure is an internal body structure, such as an internal organ, e.g., heart, kidney, stomach, lung, intestines, and etc. The first method may be used in a variety of different kinds of animals, where the animals may be “mammals” or “mammalian,” where these terms are used broadly to describe organisms which are within the class mammalia, including the orders carnivore, e.g., dogs and cats, rodentia, e.g., mice, guinea pigs, and rats, lagomorpha, e.g. rabbits, and primates, e.g., humans, chimpanzees, and monkeys. In many applications, the subjects or patients will be humans.

In many representative alternate applications of the first method, the tissue location is a cardiac location. As such and for ease of further description, the various aspects of the first method are now reviewed in terms of evaluating motion of a cardiac location. The cardiac location may be endocardial, epicardial, or a combination of both, as desired, and may be an atrial location, a ventricular location, or a combination of both. Where the tissue location is a cardiac location, in representative applications of the first method, the cardiac location is a heart wall location, e.g., a chamber wall, such as a ventricular wall, a septal wall, etc. Although the invention is now further described in terms of cardiac motion evaluation applications, the invention is not so limited, the invention being readily adaptable to evaluation of movement of a wide variety of mechanical systems, equipment control systems, robotics, as well as various tissue locations.

In practicing applications of the first method, one or more multi-electrode leads are located relative to a human or a mammalian body, i.e., a “target body”. One or more multi-electrode leads may be implantable such that leads deliver an electromagnetic energy pulse within the body, or alternately from locations outside of the body.

In one aspect of the first method, a system may be employed that includes at least one lead having multiple programmable satellites. Each satellite comprises at least two electrodes is stably associated with a cardiac location of interest, e.g., a heart wall, such as a ventricular wall, septal wall, etc., such that energetic pulse and waveform detections by the sensing element can be correlated with movement of the cardiac location of interest.

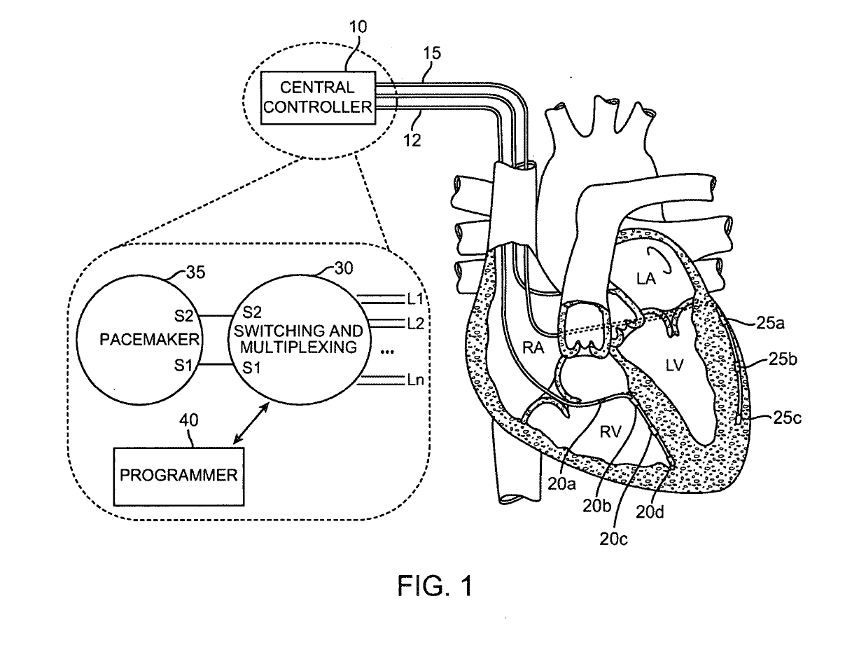

FIG. 1 is a high level schematic of a cardiac pacing and signal detection system in which a number of satellite units (or satellites) are disposed on one or more pacing leads and communicate with a pacing and detection controller 10, typically referred to as the central controller. Central controller 10 provides extra-cardiac communication and control elements for the overall system of FIG. 1, and may include, for example, a pacing can of a pacemaker, typically implanted under a subject's skin away from the heart. In the specific configuration illustrated, there are three pacing leads, including a right ventricular lead 12 and a left ventricular lead 15.

Right ventricular lead 12 emerges from the central controller, and travels from the subcutaneous location of the central controller into the subject's body (e.g., preferably, a subclavian venous access), and through the superior vena cava into the right atrium. From the right atrium, right ventricular lead 12 is threaded through the tricuspid valve to a location along the walls of the right ventricle. The distal portion of right ventricular lead 12 is preferably located along the intra-ventricular septum, terminating with fixation in the right ventricular apex. Right ventricular lead 12 is shown as having satellites 20a, 20b, 20c, and 20d. In one optional configuration, satellite 20a includes a pressure sensor in the right ventricle.

Similarly, left ventricular lead 15 emerges from central controller 10, following substantially the same route as right ventricular lead 12 (e.g., through the subclavian venous access and the superior vena cava into the right atrium). In the right atrium, left ventricular lead 15 is threaded through the coronary sinus around the posterior wall of the heart in a cardiac vein draining into the coronary sinus. Left ventricular lead 15 is provided laterally along the walls of the left ventricle, which is a likely position to be advantageous for bi-ventricular pacing. Left ventricular lead 15 is shown as having satellites 25a, 25b, and 25c.

The number of satellites 20a, 20b, 20c, 20d, 25a, 25b, and 25c shown is but one example. In some versions, there may be more; in others, fewer. The particular implementation described below allows a large number of individually addressable satellites and/or individually addressable electrodes. A typical exemplar lead may provide four electrodes per satellite and eight satellites per lead. A signal multiplexing arrangement, according to certain aspects of the present invention, facilitates including active devices to a lead for pacing and signal collection purposes, e.g., right ventricular lead 12. As mentioned above and described below in detail, the electrodes controlled by the satellites may be used for pacing, and may also be used to detect analog signals, such as local analog cardiac depolarization signals.

Central controller 10 is shown in an enlarged detail to be a distributed system, where multiplexing and switching capabilities are provided by a switching and multiplexing circuit 30 that augments a pacemaker 35 (commonly referred to as a pacemaker “can”), which may be any conventional pacemaker. The switching circuit acts as an interface between the pacemaker and a plurality of leads, designated L1 . . . Ln. Right and left ventricular leads 12 and 15 are examples of such leads, which are configured for placement within the heart in an arrangement and by procedures well known by those skilled in the art. The arrangement described above with respect to leads 12 and 15 is representative.

Switching and multiplexing circuit 30 may be housed within a can similar to that of pacemaker 35, which housing is configured for implantation in the subject adjacent to pacemaker 35. Switching and multiplexing circuit 30 is electrically coupled to pacemaker 35 via a pair of signal lines S1 S2, which are referenced herein as SI and S2, wherein SI represents ground and S2 is a voltage supply. These lines may be configured at the pacemaker end in the form of a connector which can be plugged into standard pacemaker lead plug receptors.

Central controller 10 performs a number of functions, which will be outlined here. The precise division of labor between switching and multiplexing circuit 30 and pacemaker 35 can be a matter of design choice. To the extent that it is desired to implement aspects of the present invention, the pacemaker can be considered to provide a power supply and the ability to generate pacing pulses of desired voltage and duration. For purposes of this discussion, switching and multiplexing circuit 30 will be described as providing the additional functionality. This is not critical, and indeed the pacemaker and the switching circuit can be implemented within a single housing.

In short, switching and multiplexing circuit 30 multiplexes the pacemaker signals among the various leads, although some signals may go to multiple leads. The switching circuit also sends signals to, and receives signals from, the satellites on the bus. At various times, the switching circuit may be used to transmit address information from the central controller to the satellites, send configuration information from the central controller to the satellites to configure one or multiple electrodes associated with selected satellites, provide power to operate the digital logic circuits within the satellite chip, transmit activation pulses from the pacemaker to the satellites, receive analog signals from the satellites, and receive digital signals, e.g., signals confirming the configuration, from the satellites.

Additionally, switching and multiplexing circuit 30 provides a communication link to external devices, such as a programmer 40, which can remotely control and program the switching circuit with operating or functional parameters, certain parameters of which can then be communicated to pacemaker 35 by the switching circuit. While any mode of telemetry may be used to transfer data between switching and multiplexing circuit 30 and programmer 40, one suitable mechanism for use with implantable devices is electromagnetic coils, where one coil is provided in switching and multiplexing circuit 30 and another is provided in programmer 40. By placing the programmer in close proximity to the subject's chest in the vicinity of the implanted switching can, telemetric communication can be established.

Information transmitted between switching and multiplexing circuit 30 and programmer 40 is in the form of AC signals which are demodulated to extract a bit stream representing the digital information to be communicated. The signal(s) transmitted by programmer 40 and received by switching and multiplexing circuit 30 provides a series of commands for setting the system operating parameters. Such operating or functional parameters may include, but are not limited to,

assignment of the electrode states, the pulse width, amplitude, polarity, duty cycle and duration of a pacing signal, the number of pulses per heart cycle, and the timing of the pulses delivered by the various active electrodes.

The AC signals sent from the programmer to the switching circuit can also provide a system operating current which can be used to power up the circuit components. To this end, the switching circuit can be provided with a rectifier bridge and a capacitor. In typical situations, the switching circuit gets its power from pacemaker 35, but could be provided with a separate battery if desired.

In addition to downloading information from a programming device, the switching circuit may also be configure to upload information such as sensing data collected and stored within a memory element of the switching circuit. Such sensing data may include, but is not limited to, blood pressure, blood volume, blood flow velocity, blood oxygen concentration, blood carbon dioxide concentration, wall stress, wall thickness, force, electric charge, electric current and electric conductivity.

The switching circuit may also be capable of storing and transmitting data such as cardiac performance parameters, which are calculated by it or the pacemaker from the sensed data. Such cardiac performance parameters may include, but are not limited to, ejection fraction, cardiac output, cardiac index, stroke volume, stroke volume index, pressure reserve, volume reserve, cardiac reserve, cardiac reserve index, stroke reserve index, myocardial work, myocardial work index, myocardial reserve, myocardial reserve index, stroke work, stroke work index, stroke work reserve, stroke work reserve index, systolic ejection period, stroke power, stroke power reserve, stroke power reserve index, myocardial power, myocardial power index, myocardial power reserve, myocardial power reserve index, myocardial power requirement, ejection contractility, cardiac efficiency, cardiac amplification, valvular gradient, valvular gradient reserve, valvular area, valvular area reserve, valvular regurgitation, valvular regurgitation reserve, a pattern of electrical emission by the heart, and a ratio of carbon dioxide to oxygen within the blood.

Switching and multiplexing circuit 30 may also function as part of a satellite power management system. As will be described in greater detail below, each satellite has a capacitor that stores sufficient charge to power certain parts of the satellite circuitry, e.g., latches storing satellite configuration information, when power is not being provided over the bus. While leakage currents may be extremely low, and normal signaling and pacing may provide enough power to keep the capacitor charged, switching circuit may be configured to periodically supply a sufficiently high voltage pulse for a few microseconds, possibly from 10 to 20 microseconds, to recharge all the satellite capacitors. Additionally, switching and multiplexing circuit 30 can be programmed to periodically, e.g., once daily, refresh the then current satellite configuration that had been stored memory. In case of a power glitch which disrupts the electrode status, switching and multiplexing circuit 30 can reset the electrode capacitors to the last configuration stored in memory.

Another function which may be performed by switching and multiplexing circuit 30 is that of transmitting analog signals from the satellites to pacemaker 35. For example, where the pacemaker is attempting to sample voltages at a plurality of locations within the heart in order to generate a map of the heart's electrical potentials, switching and multiplexing circuit 30 enables this by providing high-speed switching between the electrodes selected for the voltage sampling.

More specifically, over a very short time period, on the order of milliseconds, the electrical potential at a selected electrode is sampled, information regarding the analog voltage is sent to pacemaker 35, and the sequence is repeated for another selected electrode. The faster the switching, the more accurate the “snap shot” of potentials is at various locations about the heart, and thus, the more accurate the electrical potential map.

In some applications, the information regarding the analog voltage is the analog signal itself. That is, the measured potentials are provided as analog signals which are carried from the satellite electrodes to pacemaker 35 by way of switching and multiplexing circuit 30 where the signal from one electrode is provided on line S 1 and the signal from another electrode is provided on line S2. An amplifier or voltage comparator circuit within pacemaker 35 may then compare the two analog voltages signals. Based on this comparison, pacemaker 35 will reconfigure the pacing parameters as necessary. Alternatively, each satellite chip could include an analog-to-digital converter that digitizes the analog voltage signal prior to sending it to switching and multiplexing circuit 30. It is believed that providing this additional functionality in the satellites would require larger satellite chips, would be more power consumptive, and would be slower since the time necessary for the charges on the capacitors in the satellites to settle and become balanced would be far greater.

Still yet, switching and multiplexing circuit 30 may function as an analog-to-digital and digital-to-analog conversion system. A sensing protocol, either programmed within switching and multiplexing circuit 30 or otherwise transmitted by an external program by programmer 40, in the form of digital signals is converted to an AC signal by switching and multiplexing circuit 30. These analog signals include current signals which drive sensing electrodes or other types of sensors, e.g., transducers; to enable them to measure physiological, chemical and mechanical signals, e.g., conductance signals, within the subject's body. The measured signals, also in analog form, are then converted to digital signals by switching and multiplexing circuit 30 and stored in memory, used to calculate other parameters by the switching circuit or transmitted to pacemaker 35 and/or programmer 40 for further processing.

A multiple electrode lead allows for greater flexibility in lead placement, as at least one of the multiple electrodes will be optimally positioned to pace the heart. Determining which of a lead's electrodes is best positioned to obtain or provide an accurate signal to and from a target tissue site or area, e.g., specific heart tissue, may be determined experimentally by controlled pacing of the heart and measuring the resulting threshold voltage of each electrode, wherein the electrode with the lowest threshold voltage is the most optimally positioned electrode for that satellite unit. Additionally, electrode(s) proximal to untargeted tissue sites or areas, e.g., the phrenic nerve, may be selectively identified, may remain inactivated, may be selectively inactivated, etc.

Once electrode(s) on each satellite unit with the lowest threshold or least sensitive to untargeted tissue sites/areas is established, then the various satellite units may be selected one at a time or in combinations to determine which satellite unit(s) and/or individual electrode configuration produces the best hemodynamic response. This latter optimization may be performed with feedback from an external device such as an ultrasound system, or with one of the other feedback systems referenced in the above published applications.

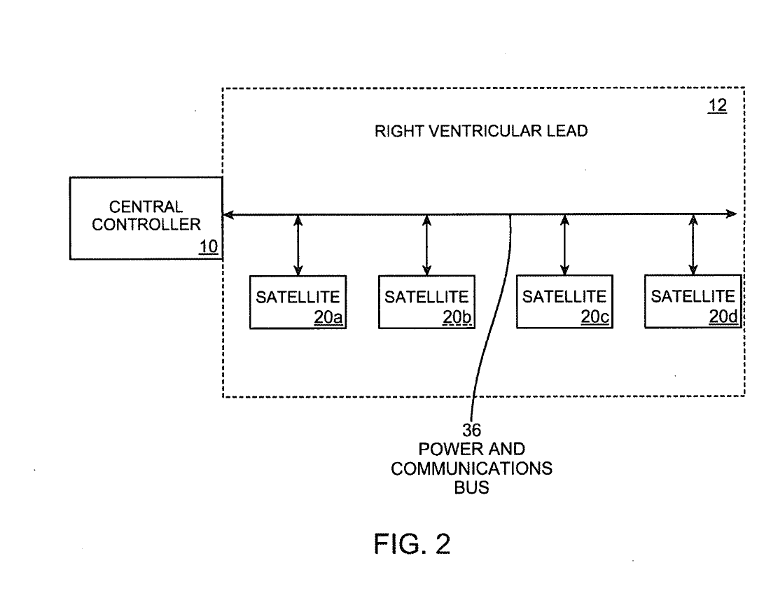

Referring now generally to the Figures and particularly to FIG. 2, FIG. 2 is a detailed schematic of the exemplary right ventricular lead 12 including four satellites 20a, 20b, 20c and 20d that are each bi-directionally communicatively coupled with a power and communications bus 36. The power and communications bus 36 comprises and represents ground S1 and the voltage supply line S2. The power and communications bus 36 is detachably connected to the central controller 10 and provides bi-directionally communicatively coupling between the central controller 10 and the four satellites 20a, 20b, 20c and 20d, and additionally providing a pathway for cardiac pacing pulses as delivered from the central controller to the ventricular lead 12.

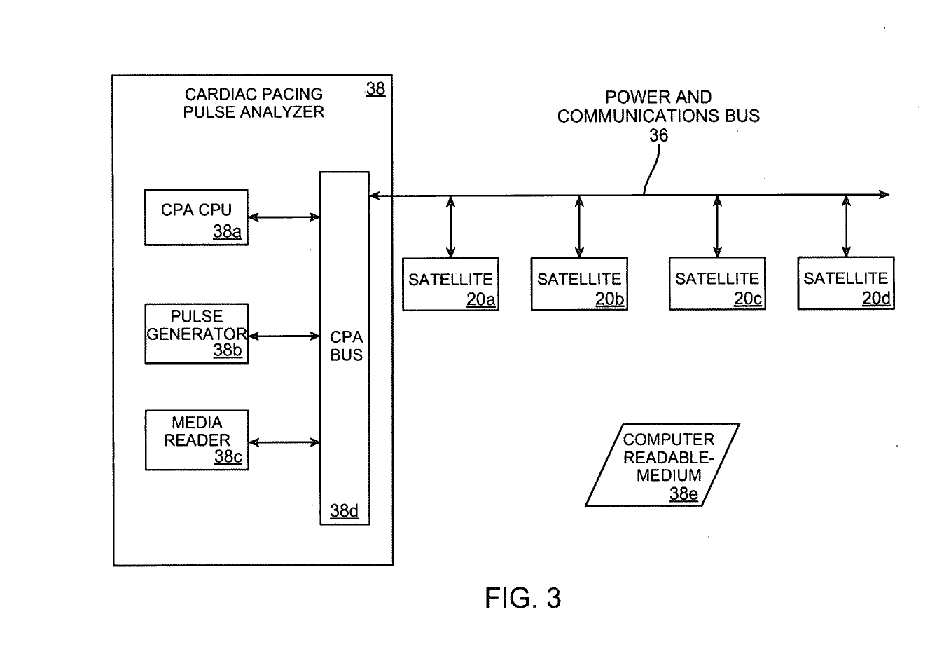

Referring now generally to the Figures and particularly to FIG. 3, FIG. 3 is a detailed schematic of a legacy cardiac pacing pulse analyzer 38 comprising an internal central processing unit 38a (hereinafter “CPA CPU” 38), a pulse generator 38b, and a media reader 38c. A cardiac pacing pulse analyzer power and communications bus 38d (hereinafter, “CPA BUS” 38d) is detachably coupled with the power and communications bus 36 of the right ventricular lead 12 and bi-directionally communicatively couples the four satellites 20a, 20b, 20c, and 20d of the right ventricular lead 12 with the CPA CPU 38a and the media reader 38c, as well as providing a pathway for cardiac pulses from the pulse generator 38b to the four satellites 20a, 20b, 20c, and 20d of the right ventricular lead 12.

The media reader 38c and the computer-readable media 38e are selected to enable the media reader 38c to read software encoded, machine executable commands from storage on the computer-readable media 38d that instantiate on or more steps or aspects of the method of the present invention.

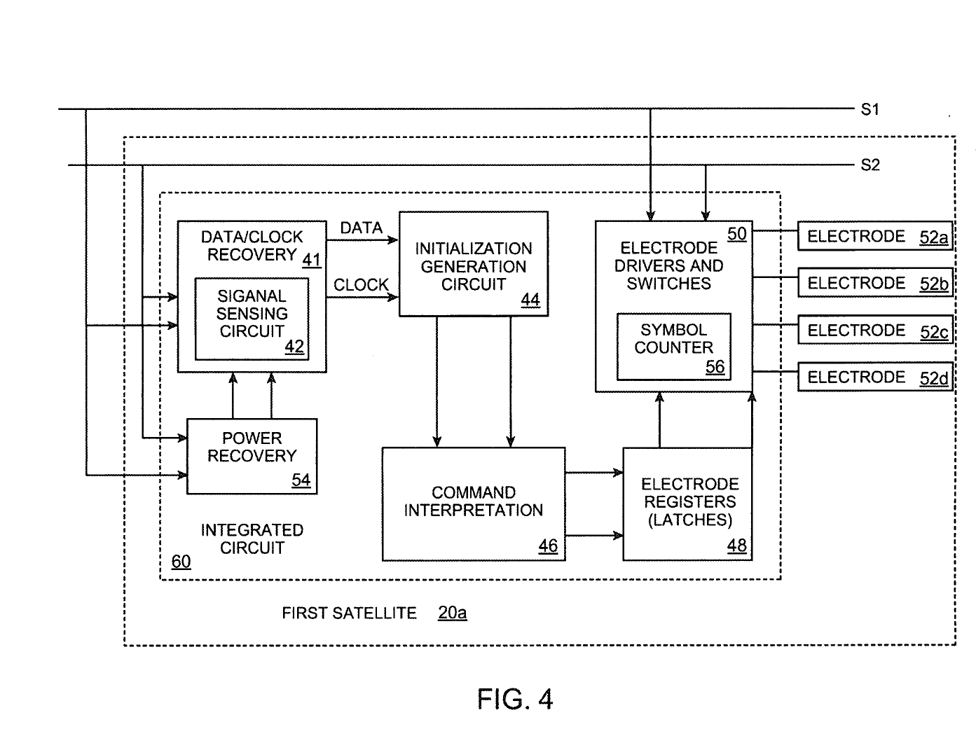

Referring now generally to the Figures and particularly to FIG. 4, FIG. 4 is a detailed schematic of the first satellite 20a of the right ventricular lead 12. A data and clock recovery circuit 41 is coupled to the ground line S1 and the voltage supply line S2 to accept signals and electrical power sent from either the central controller 10 or the cardiac pacing pulse analyzer 38. A signal sensing circuit 42 examines the amplitude and voltage level of electrical pulses received from the ground line S1 and the voltage supply line S2. Results of the processing of the data and clock recovery circuit 41, to include the processing of the signal sensing circuit 42 are transmitted to an initialization generation circuit 44. The initialization generation circuit 44 activates a ground line S1 and the voltage supply line S2.

The command interpretation circuit 46 directs a plurality of electrode registers 48 and electrode drivers and switches circuit 50 in accordance with an interpretation of pulses received from the ground line S1 and the voltage supply line S2. The setting of the electrode drivers and switches 50 determines which, if any, of the electrodes 52a, 52b, 52c and 52d shall transfer a cardiac pacing pulse received from the ground line S1 and the voltage supply line S2 and to a living tissue, such as the heart of FIG. 1. The cardiac pacing pulse or pulses may be received from the ground line S1 and the voltage supply line S2 from either the central controller 10 or the cardiac pacing pulse analyzer 38. A power recovery circuit 54 stores electrical power received from the ground line S1 and the voltage supply line S2 and supplies the elements 40-56 of the first satellite 20a with the stored electrical power.

The first ventricular lead 12 may apply a differential 4-state technique to quickly set the electrodes 52a, 52b, 52c and 52d into one of 16 states when first ventricular lead 12 is connected to the cardiac pacing pulse analyzer 38 and provides a more complete level of functionality when connected to the central controller 10.

The first ventricular lead 12 may be in a default state when first unpackaged and connected to the cardiac pacing pulse analyzer 38. When a 2 V pacing pulse is transmitted through either the ground wire S1 and the voltage wire S2, or alternatively a single wire and a RV coil (not shown), the most distal satellites 20c and 20d of the first ventricular lead 12 become a cathode and an anode, respectively and the proximal two satellites 20a and 20b are turned off.

A wake-up command may be sent from either the cardiac pacing pulse analyzer 38 or the central controller 10. On receipt of a wake-up command by the first satellite 20a, the switches of the electrode drivers and switches circuit 50 are turned off, which minimizes charge imbalance on the electrodes 52a, 52b, 52c and 52d and reduces variations caused by varying electrode impedances or polarization. Current sources and comparators of the first satellite 20a are enabled.

When a pulse received by the first satellite 20a is longer than 60 microseconds, which will be typical of most cardiac pacing pulses, communication capacitors of the first satellite 20a are reset to zero, the switches of the electrode drivers and switches circuit 50 are connected according to their stored configuration, a symbol counter 56 is set to 00, and then the first satellite 20a goes to sleep, wherein current sources and comparators are disabled.

The communication protocol of the satellites 20a, 20b, 20c and 20d in the default state is a combination of pulse width modulation and amplitude modulation, arranged to be self-referencing. Two pulses are needed to set two bits. Each pulse may be either twenty microseconds or forty microseconds in duration and either three Volts or five Volts in amplitude. A second following pulse may be the complement of the first pulse. Thus, there are may be four symbols created with two pulses as shown in Table A:

TABLE A

| | First pulse | | second pulse | |
|---|---|---|---|---|
| | width | Amplitude | width | Amplitude |
| Symbol | (microseconds) | (v) | (microseconds) | (v) |
| W | 20 | 3 | 40 | 5 |
| X | 20 | 5 | 40 | 3 |
| Y | 40 | 3 | 20 | 5 |
| Z | 40 | 5 | 20 | 3 |

It is expected that this symbol system will be realized using four capacitors C00, C01, C10 and C11 to store four voltages, which are then compared using two comparators; the command interpretation circuit 46 then interprets the transmitted symbol. Two of the capacitors will be integrating a current source during each pulse. The current source output does not vary significantly with supply voltage.

On the first pulse, the symbol counter 56 will be 000, and a C00 timing capacitor will integrate the current from the current source for the duration of the pulse. When the pulse ends, the current source goes to sleep and the C00 timing capacitor is disconnected from the current source. While the pulse is high, an amplitude capacitor C10 is connected to the voltage line S2 via a resistor that allows full charging in about 10 microseconds. The symbol counter 56 may then be incremented by one state.

On a second following rising edge, the current source and comparators of the first satellite 20a are turned on and a C01 timing capacitor integrates the current source. An amplitude capacitor C11 stores the voltage from the voltage line S2 and is clipped in a manner similar to that of the first pulse.

While the second pulse is integrating, a first comparator is comparing the voltages stored on timing cap C00 to timing cap C01 and a second comparator is comparing the voltage stored on the amplitude capacitor C10 to the amplitude capacitor C11. The results may be latched on the falling edge of the second pulse onto a timing flip flop FF0 and an amplitude flip flop FF1. Logic is used to decode the two states of these two flip flops to represent symbol A as either W, X, Y or Z.

After the falling edge of the second pulse, the four capacitors C00, C01, C10 and C11 may all be discharged to zero using ripple logic. And the symbol counter 56 may be advanced one state.

A similar sequence occurs for a third and a fourth the pulse, setting a second flip flop circuit FF2 and a third FF3 flip flop circuit to represent symbol B. Throughout these four pulses, the switches are turned off. In addition, if any of these pulses exceeds a pre-determined standard duration, for example in asserting a sixty microseconds pulse duration as a standard for pulse duration comparison, the capacitors C00, C01, C10 and C11 may be discharged and the symbol counter may be reset to 000.

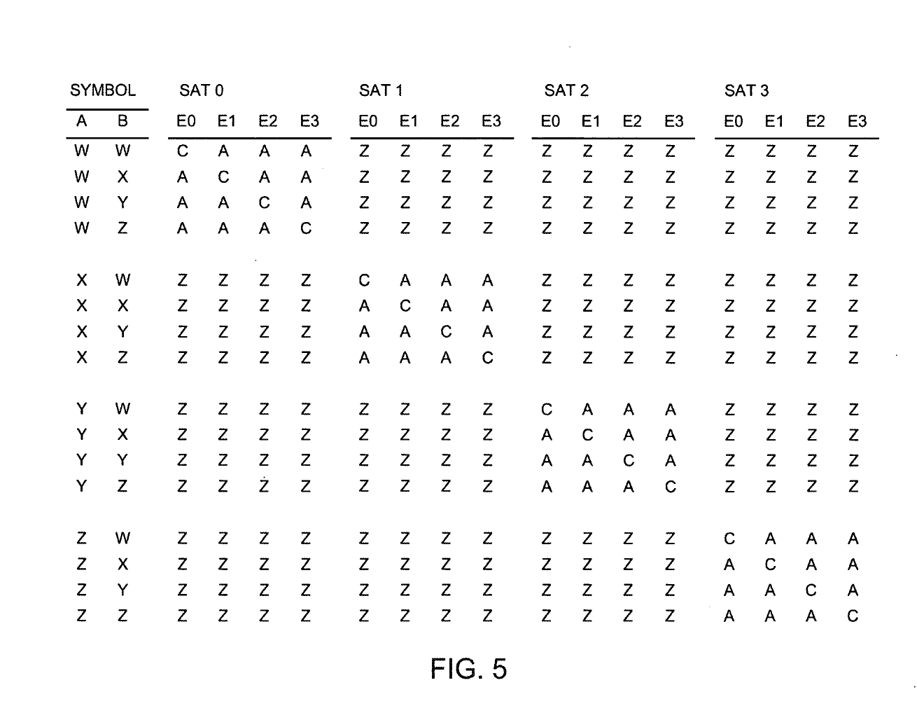

On the fifth pulse, the symbol counter 56 may read 100, indicating that all four pulses were less than 60 microseconds. The first symbol represents the satellite 20a being enabled wherein the three remaining satellites 20b, 20c and 20d are disabled. The second symbol represents the electrode 52a, 52b, 52c and 52d on the enabled satellite 20a, 20b, 20c and 20d that is to be connected as cathode; the remaining electrodes electrode 52a, 52b, 52c and 52d on the selected satellite 20a, 20b, 20c and 20d are to be connected as anode. With a symbol count of 100, the switch configuration will be set according to FIG. 5.

The fifth pulse may be the pacing pulse; in any event the fifth pulse may be at least 60 microseconds in duration. Once the 60 microsecond's threshold is reached, the new configuration will be used to enable the appropriate switches, the four capacitors C00, C01, C10, C11 will be discharged and the symbol counter 56 may be reset to 000.

Note that the comparators need to be enabled during the second and fourth pulses, when the value of the symbol counter 56 would respectively 001 and 011, and the current sources need to be enabled during the first four pulses, i.e., values of the symbol counter 56 of 000, 001, 010, and 011. Also note that the expected time between the four pulses is about 20 milliseconds when programmed using the cardiac pacing pulse analyzer 38. When this protocol is invoked by central controller 10, the time between pulses may be as short as 5 microseconds

When the central controller 10 is implanted in a living being, it is desirable to modify the communication protocol somewhat. In order to prevent the command interpretation circuit 46 from waking up during each pacing pulse during normal operation, a high frequency wakeup signal is supported by the first modality. For example, by communicating six pulses of five microseconds each, the right ventricular lead 12 maybe alerted to interpret commands and data received from the power and communications bus 36 in accordance with the first modality.

It is understood that certain optional aspects of the command interpretation circuit 46, the command interpretation circuit 46 may be programmed or configured to apply three or more communications modalities, whereby pulses received by and sent from the first satellite 20a may be formatted and interpreted by the right ventricular lead 12 in accordance with one modality selected from a plurality of communications modalities.

According to other aspects of the invention, the same symbol generation scheme may be as described in the Table B above. It may be desirable to shorten the time for communication by reducing the pulse widths, for example, from a range of twenty microseconds to forty microseconds to a range of two microseconds to four microseconds. The time between pulses may also be considerably shorter, and likely determined by noise considerations.

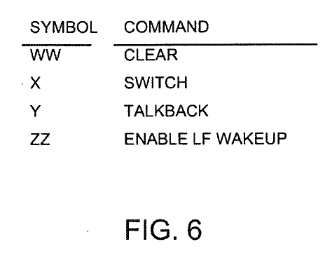

It may be desirable to support additional commands in accordance with the first modality. Following a high frequency wake-up pulse a first symbol and a second symbol will have the meanings to the first electrode 20a as presented in FIG. 6.

A clear command may set the switches of the electrode drivers and switches 50 to an off, or high impedance, state. To ensure robust communication of this command, two “W” symbols preceded by a HF Wakeup signal enables the Clear command. It would be enforced on the first pulse following the second “W” symbol.

For test purposes and also for backup implanted communication, a low frequency wakeup signal may be enabled by sending a high frequency wake-up command followed by two “Z” symbols. Following the generation of this command, the communication protocol will be in the second modality. The electrode configuration is not changed by sending this command. The high frequency wake-up command remains enabled following the command.

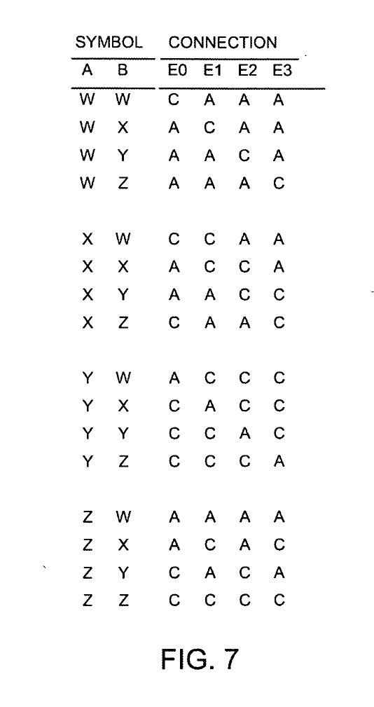

When the “X” symbol follows the high frequency wake-up signal, the next symbol represents the satellite being switched, as before wherein W=Sat0 20A, X=Sat 1 20b, Y=Sat 2 20c, Z=Sat 3 20d. The second and third symbols of a command determines which electrodes 52a, 52b, 52c and 52d on the selected satellite 20a, 20b, 20c and 20d are anodes and which are cathodes as presented below in FIG. 7.

Thus, a high frequency wake-up signal followed by an XYWW would set E0 52a to a cathode and E1-E3 52b, 52c and 52d to anode on Sat 2 20b. This switch command can be abstracted as high frequency wake-up signal followed by XABC, where A determines the satellite 20a, 20b, 20c and 20d and BC determine the configuration of the electrodes 52a, 52b, 52c and 52d.

A talkback command issued by the central controller 10 queries a specific satellite 20a, 20b, 20c, and 20d for a current configuration setting. Two symbols are needed to send the command, wherein “Y” is the command and the next symbol represents the satellite 20a, 20b, 20c, and 20d being queried. Thus, “YW” queries Sat 0 20a, “YX” queries Sat1 20b, “YY” queries Sat 2 20c, and “YZ” queries Sat 3 20d.

The signaling requesting a talkback response may be or comprise a differential current between two adjacent pulses, wherein the right ventricular lead 12 circuit may pull down extra current either during the first of two pulses or during the second of two pulses.

In certain applications of the present invention, in accordance with the second modality, pacing pulses generated by the cardiac pacing pulse analyzer 38 may be any amplitude between 0.5 volts and 10.0 volts, and the cardiac pacing pulse analyzer 38 may skip a pacing pulse to issue a command to the first ventricular lead 12, wherein communication between the cardiac pacing pulse analyzer 38 and the first ventricular lead 12 will occur during the refractory window of the heart in six pulses and within approximately a 110 millisecond time period.

The commands issued by the cardiac pacing pulse analyzer 38 may comprise pulses that may be, in one exemplary optional aspect of method of the present invention, nominally twenty microseconds to 160 microseconds and possibly separated by two microseconds in accordance with the first modality, and wherein the pulses may be separated by 20 milliseconds in accordance with the second modality. The proposed pulse widths have 33% margin detection for PVT/noise, and commands having pulses in the ranges 20-80-320-1280 uSec may increase the margin detection to 100%.

The commands issued by the central controller 10 and the cardiac pacing pulse analyzer 38 and in accordance with the second modality and transmitted to the leads 12 and 15 may be constructed of various components, to include Wakeup−>Start Bit−>Command+data payload−>Drive in−>Sleep. These components and their function are described below.

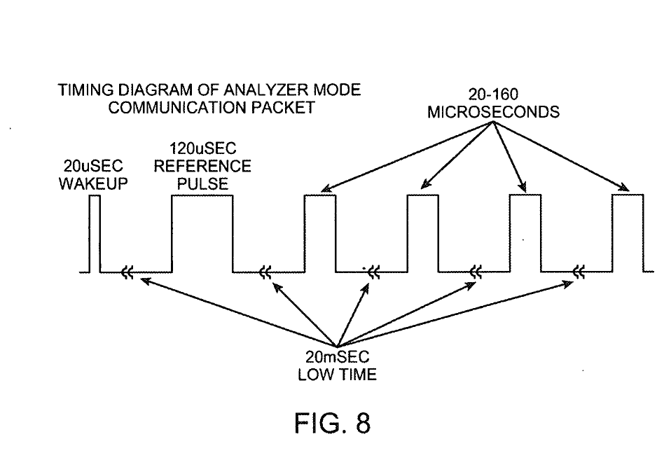

Referring now to FIG. 8, a timing diagram of a sample command formatted by the cardiac pacing pulse analyzer 38 in accordance with the second modality analyzer mode data packet is illustrated.

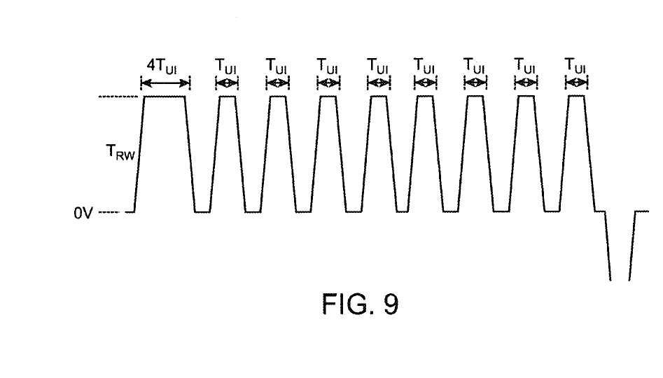

Referring now to FIG. 9, a high frequency wakeup command in accordance with the first modality and as generated by the central controller 10 may include a period of four Unit Intervals (hereinafter “UI”) of 0.7 microseconds duration at V_{HI} followed by 8 cycles from 0V to V_{HI} with a period of two unit intervals, followed by an optional charge balance pulse.

A start bit of a command may indicate a start of command and may serve as a sync bit. According to an additional aspect of the method of the invention, a 20 microsecond pulse may comprise a start bit, and may simultaneously serve as a low frequency wakeup signal in analyzer mode. Alternatively a 120 microsecond reference pulse at a V_{HI} voltage may be employed as a start bit.

One or more data pulses of a command may be defined by one of four possible durations of twenty microseconds, forty microseconds, eighty microseconds, or 160 microseconds at V_{HI} voltage. The value of each data pulse may be determined by separately comparing each data pulse to the reference pulse duration as received by a satellite 20a, 20b, 20c and 20d divided by two and/or four. In accordance with the first modality, data pulse duty cycles may be greater than fifty percent.

A drive-in signal may be communicated by a falling edge of a last or sixth pulse of a command, wherein the drive-in signal determines when s command will be executed by a receiving satellite 20a, 20b, 20c and 20d.

As presented in Table B, the commands executable by the satellites 20a, 20b, 20c and 20d that are supported in both the first modality, or “device mode”, and the second modality (or “analyzer mode”) are indicated in Table C below with an X indicator. Commands supported only by the device mode are indicated by a one value, and commands supported only by the analyzer mode are indicated by a zero value.

TABLE B

| Comms Mode | Value |
|---|---|
| Analyzer | 0 |
| Device | 1 |
| Both Analyzer and Device | X |

Pulse and bit definitions are provided in the Table C below.

TABLE C

| | Pulse | | | Decoded bits | | Comms |
|---|---|---|---|---|---|---|
| Pulse # | Width | Symbol | Function | MSB | LSB | Mode |
| 0 | 20 uSec | | Analyzer | N/A | N/A | 0 |
| | | | Wakeup | | | |
| 1 | 120 uSec | R | Reference | N/A | N/A | X |
| 2-5 | 20 uSec | W | Data | 0 | 0 | X |
| 2-5 | 40 uSec | X | Data | 0 | 1 | X |
| 2-5 | 80 uSec | Y | Data | 1 | 0 | X |
| 2-5 | 160 uSec | Z | Data | 1 | 1 | X |

Talkback Only Device Mode

| | | | | | | |
|---|---|---|---|---|---|---|
| 6-21 | 20 uSec | T | Talkback Data | N/A | N/A | 1 |
| | | | Bits | | | |

Switch Only Device Mode

| | | | | | | |
|---|---|---|---|---|---|---|
| 6-9 | 20 uSec | W | Switch Config | 0 | 0 | 1 |
| 6-9 | 40 uSec | X | Switch Config | 0 | 1 | 1 |
| 6-9 | 80 uSec | Y | Switch Config | 1 | 0 | 1 |
| 6-9 | 160 uSec | Z | Switch Config | 1 | 1 | 1 |

It is understood that six pulses shown as pulse zero through five in the Table C above define most commands to the satellites 20a, 20b, 20c, and 20d. In the first modality, or device mode, switch and talkback commands can use up to ten or twenty two pulses respectively as shown in Table C.

Some or all commands may be decoded as two bits per pulse. For transmission from the first satellite 20a to the central controller 10, talkback data bits are encoded as one bit for every two pulses.

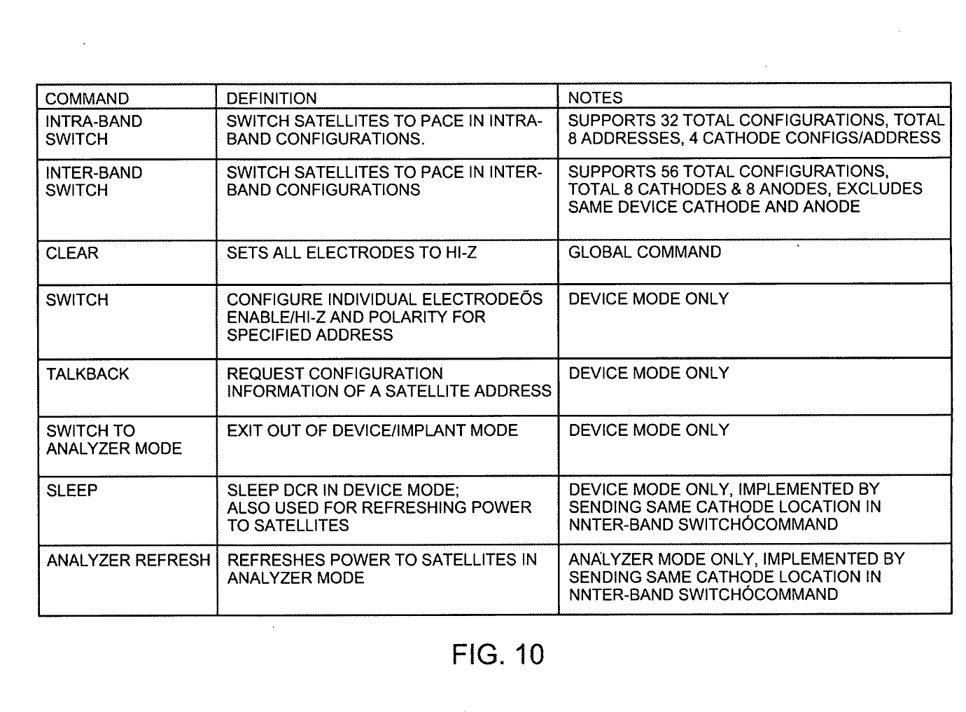

Referring now to FIG. 10, FIG. 10 illustrates that the structure of commands may vary between the first modality and the second modality, whereas messages issued from the central controller and formatted in accordance with the first modality, i.e., device mode, may include a high frequency wakeup signal, a start signal, a reference signal, a command, and a sleep signal. Alternatively, messages issued from the cardiac pacing pulse analyzer 38 are formatted in accordance with the second modality and may include a wakeup signal, a reference signal and a command

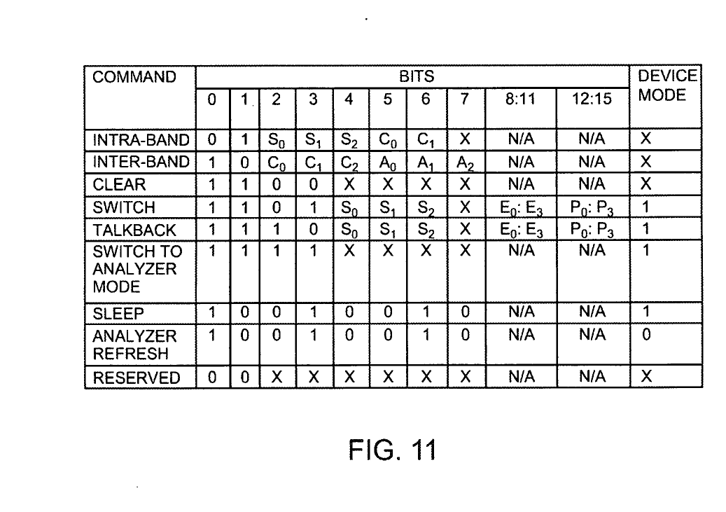

Referring now to FIG. 11, FIG. 11 illustrates command encoding, wherein
S₀₋₂—Satellite address, 3 bits provide total of 8 addresses
C₀₋₁—Cathode Location, 2 bits provide total of 4 possible quadrant cathode locations for given Satellite address in intra-band configurations
C₀₋₂—Cathode address, 3 bits provide total of 8 Cathode addresses for inter-band configuration
A₀₋₂—Anode address, 3 bits provide total of 8 Cathode addresses for inter-band configuration
E₀₋₃—Electrode Enable
   1=Enable Electrode
   0=Disable Electrode and Make it High-impedance
P₀₋₃—Electrode Polarity
   1=Connected to Anode/S2
   0=Connected to Cathode/S1

The talkback command and response is supported only in the first modality and when the central controller is coupled with a lead 12 and 15. A talk back command requires additional “talkback data” pulses of twenty microseconds nominal duration to transmit a satellite configuration to the central controller 10. The pulses six through twenty-one during a talkback command act may as return data pulses carrying information from a satellite 20a, 20b, 20c and 20d to the key controller 10.

Two pulses may transmit one bit of information ion a talkback command and in accordance with the first modality. For example, a first talkback bit may be transmitted by pulses six and seven, and a second talkback bit may be transmitted by pulses eight and nine and so on.

To transmit a zero value a satellite 20a, 20b, 20c and 20d addressed by a talkback command may pull down on odd numbered pulse against a high impedance resistor, whereas to transmit a one value a satellite 20a, 20b, 20c and 20d may pull down on even numbered pulse. Received data is decoded by comparing currents during even and odd pulses. Received data is defined as

Bit 0—Even pulse current<odd pulse current (e.g. I(6)<I(7))

Bit 1—Even pulse current>odd pulse current (e.g. I(6)>I(7))

Nominal duration for the talk back command is 750 microseconds assuming duty cycles greater than fifty percent.

In accordance with the first modality, each lead 12 and 15 may sleep after a sleep command is received via the power and command bus 36, and each lead may be refreshed by receipt of a wake-up command or upon completion of a sleep sequence.

In the second modality the lead 12 and 15 may sleep after completion of a command and may refresh after receipt of a cardiac pacing pulse or a refresh command.

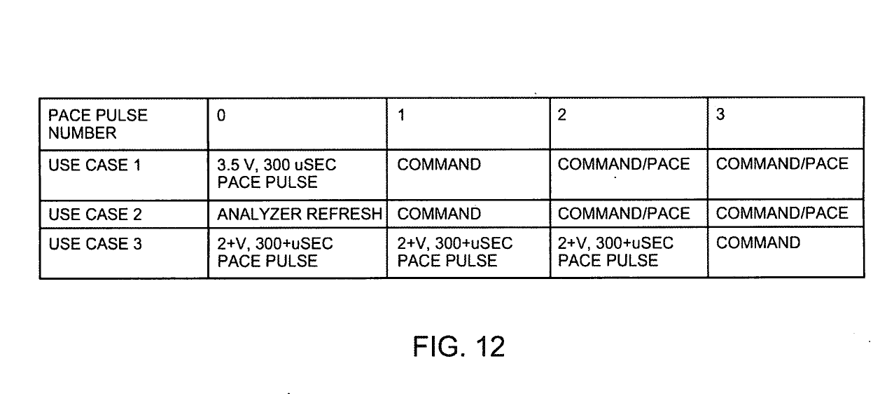

Referring now to FIG. 12, programming a new lead 12 and 15 from a completely discharged state a power up is required before communication can be sent to the new lead 12 and 15. A power up of a lead 12 and 15 can be achieved by either providing (a.) one 3.5 Volt, 300 microsecond pacing pulse; (b.) 3 pace pulses of greater than 2 Volts and greater than 300 microseconds; or (c.) or providing a refresh command before sending a communication pulse in accordance with the second modality.

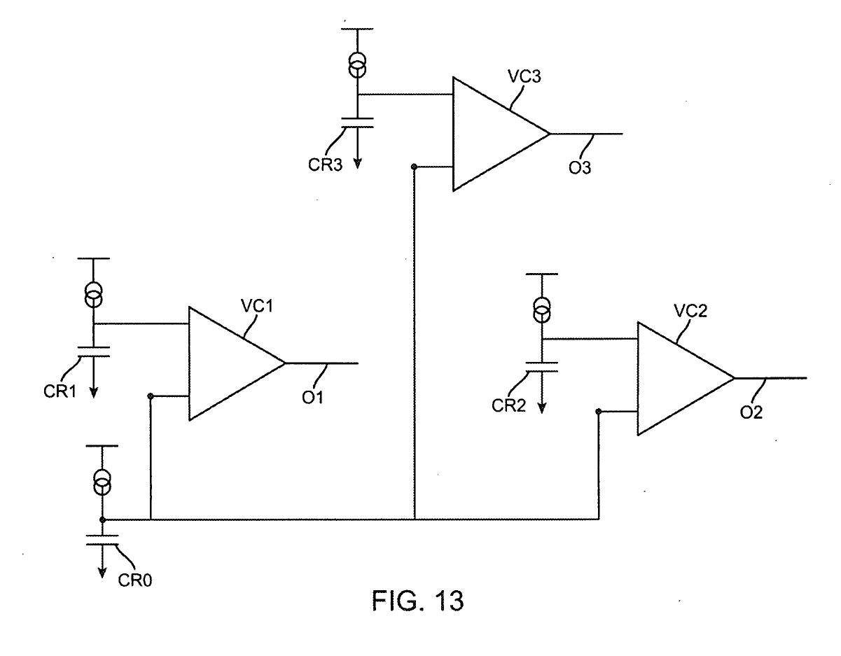

Referring now generally to the Figures and particularly to FIG. 13, according to even other aspects of the first satellite 20a, the first satellite 20a may include a plurality of reference capacitors CR0, CR1, CR2, and CR3 and a plurality of voltage comparators VC1, VC2 and VC3 of the first satellite 20a are applied to compare the time duration of data pulses of a command with a reference pulse time duration of the same command. A reference charge of a primary reference capacitor CF0 is established by applying the reference pulse of the command to the reference capacitor CF0. The use of the reference pulse of the command as measured by the first satellite 20a reduces the effect of attenuation or perturbation of the measurements performed by the first satellite 20a and imposed by variations of electrical or structural characteristics, qualities and tolerances imposed in the manufacturing, fabrication and/or assembly processes of the first satellite 20a.

After the primary reference capacitor CR0 is charged by the reference pulse, a data pulse of the same command comprising the reference pulse is then applied to charge a first reference capacitor CR1, a second reference capacitor CR2 and a third reference capacitor CR3. The charge of the first reference capacitor CR1 caused by applying the data pulse is compared to one fourth of the charge of the primary reference capacitor CR0 by a first comparator VC1, and a first comparator output value O1 of the first comparator VC1 is flipped when the charge of the first reference capacitor CR1 exceeds the one fourth of the charge of the primary reference capacitor CR0.

The charge of the second reference capacitor CR2 caused by applying the data pulse is also compared to one half of the charge of the primary reference capacitor CR0 by a second comparator VC2, and a second comparator output value O2 of the second comparator VC2 is flipped when the charge of the second reference capacitor CR2 exceeds one half of the charge of the primary reference capacitor CR0.

In addition, the charge of the third reference capacitor CR3 caused by applying the data pulse is also compared to the charge of the primary reference capacitor CR0 by a third comparator VC3, and a third comparator output value O3 of the third comparator VC3 is flipped when the charge of the third reference capacitor CR3 exceeds the charge of the primary reference capacitor CR0. The three outputs O1, O2 and O3 from the three voltage comparators VC1, VC2 and VC3 thus indicate the fractional duration of the data pulse in specific ratios to the reference pulse duration as measured by the first satellite 20a.

The reference capacitors CR0, CR1, CR2, and CR3 and the voltage comparators VC1, VC2 and VC3 may be comprised within an integrated circuit 60 of the first satellite 20a. When a 100 nano-amp current is applied at a 5.0 Volt level for 120 microseconds to charge the primary reference capacitor CR0, each reference capacitor CR0, CR1, CR2 and CR3 may function effectively at a seven Pico farad degree of capacitance. The area of the integrated circuit 60 dedicated to presenting the four reference capacitors CR0, CR1, CR2, and CR3 and the three voltage comparators VC1, VC2 and VC3 may be on the order of 3.1 percent of the cross sectional area of the integrated circuit 60.

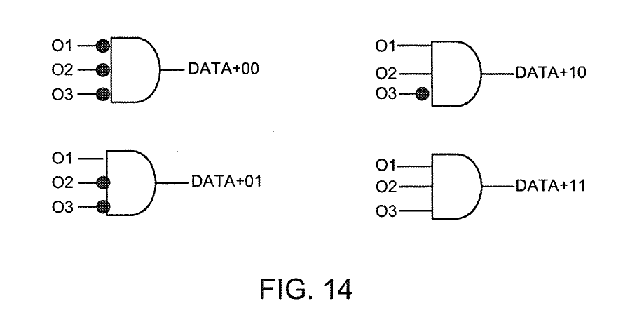

Referring now generally to the Figures and particularly to FIGS. 13 and 14, according to even other aspects of the first satellite 20a, the outputs O1, O2 and O3 of each of the three voltage comparators VC1, VC2 and VC3 are applied to an Logic Circuit 58 to extract two bits of information from a single source data pulse when processed in accordance with the method of FIG. 13. When the data pulse is measured to be less than one fourth of the reference pulse in time duration, the three outputs O1, O2 and O3 are each ZERO values and the Logic Circuit 58 presents an output representative of a 00 information content derived from the data pulse.

When the data pulse is measured to be more than one fourth, but less than one half, of the reference pulse in time duration, the three outputs values O1, O2 and O3 are ONE, ZERO and ZERO respectively, and the Logic Circuit 58 presents an output representative of a 01 information content derived from the data pulse. When the data pulse is measured to be more than one half of, but less than equal to, the reference pulse in time duration, the three outputs values O1, O2 and O3 are ONE, ONE and ZERO respectively, and the Logic Circuit 58 presents an output representative of a 10 information content derived from the data pulse. When the data pulse is measured to be greater than the reference pulse in time duration, the three outputs values O1, O2 and O3 are ONE, ONE and ONE respectively, and the Logic Circuit 58 presents an output representative of a 11 information content derived from the data pulse.

One or more aspects of the present invention may be in the form of computer-readable medium 38d having programming stored thereon for implementing the subject methods. The computer-readable media 38d may be, for example, in the form of a computer disk or CD, a floppy disc, a magnetic “hard card”, a server, or any other computer-readable media 38d capable of containing data or the like, stored electronically, magnetically, optically or by other means. Accordingly, stored programming embodying steps for carrying-out the subject methods may be transferred or communicated to a processor, e.g., by using a computer network, server, or other interface connection, e.g., the Internet, or other relay means.

More specifically, computer-readable medium 38d may include stored programming embodying an algorithm for carrying out the subject methods. Accordingly, such a stored algorithm is configured to, or is otherwise capable of, practicing the subject methods. The subject algorithm and associated processor may also be capable of implementing the appropriate adjustment(s).

The term “computer-readable medium” as used herein refers to any suitable medium known in the art that participates in providing instructions to the network for execution. Such a medium may take many forms, including but not limited to, non-volatile media, and volatile media. Non-volatile media includes, for example, optical or magnetic disks, tapes and thumb drives. Volatile media includes dynamic memory.

The methods, systems and programming of the invention may be incorporated into a variety of different types of implantable systems. Implantable systems of interest include, but are not limited to, those described in: United states application Ser. Nos. 11/664,340; 11/731,786; 11/562,690; 12/037,851; 11/219,305; 11/793,904; 12/171,978; 11/909,786; The disclosures of which are herein incorporated by reference.

While the present invention has been described with reference to the specific applications thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

The foregoing disclosures and statements are illustrative only of the present invention, and are not intended to limit or define the scope of the present invention. The above description is intended to be illustrative, and not restrictive. Although the examples given include many specificities, they are intended as illustrative of only certain possible applications of the present invention. The examples given should only be interpreted as illustrations of some of the applications of the present invention, and the full scope of the Present Invention should be determined by the appended claims and their legal equivalents. Those skilled in the art will appreciate that various adaptations and modifications of the just-described applications can be configured without departing from the scope and spirit of the present invention. Therefore, it is to be understood that the present invention may be practiced other than as specifically described herein. The scope of the present invention as disclosed and claimed should, therefore, be determined with reference to the knowledge of one skilled in the art and in light of the disclosures presented above.

1-3. (canceled)

4. A pulse delivery system comprising:
a power and communications bus;
a satellite capable of incorporeal implantation and coupled with the power
and communications bus, and comprising:
a signal sensing circuit coupled with the power and communications bus;
a command logic circuit coupled with the signal sensing circuit and programmed to distinguish and interpret at least two modalities of command and information signals; and
at least one programmable electrode coupled with the command logic, the at least one programmable electrode configured to deliver a pulse in accordance with at least one command transmitted through the power and communications bus, further comprising a default mode, wherein the at least one programmable electrode delivers a pulse in accordance with the default mode and without implementing a command transmitted through the power and communications bus.

5-16. (canceled)

17. A pulse delivery system comprising:
a power and communications bus;
a satellite capable of incorporeal implantation and coupled with the power and communications bus, and comprising:
a signal sensing circuit coupled with the power and communications bus;
a command logic circuit coupled with the signal sensing circuit and programmed to distinguish and interpret at least two modalities of command and information signals; and
at least one programmable electrode coupled with the command logic, the at least one programmable electrode configured to deliver a pulse in accordance with at least one command transmitted through the power and communications bus, wherein the at least one command includes at least one data pulse, wherein a duration and a voltage level of the at least one data pulse provide two bits of information to the satellite and the duration of the data pulse indicates a programming selection for the satellite.

18. A pulse delivery system comprising:
a power and communications bus;
a satellite capable of incorporeal implantation and coupled with the power and communications bus, and comprising:
a signal sensing circuit coupled with the power and communications bus;
a command logic circuit coupled with the signal sensing circuit and programmed to distinguish and interpret at least two modalities of command and information signals; and
at least one programmable electrode coupled with the command logic, the at least one programmable electrode configured to deliver a pulse in accordance with at least one command transmitted through the power and communications bus, wherein the duration of the data pulse indicates a programming selection for the satellite and the at least one command further comprises a second data pulse, wherein a duration and a voltage level of the at least one data pulse directs the at least one programmable lead to act as a cathode.

19. A pulse delivery system comprising:
a power and communications bus;
a satellite capable of incorporeal implantation and coupled with the power and communications bus, and comprising:
a signal sensing circuit coupled with the power and communications bus;
a command logic circuit coupled with the signal sensing circuit and programmed to distinguish and interpret at least two modalities of command and information signals; and
at least one programmable electrode coupled with the command logic, the at least one programmable electrode configured to deliver a pulse in accordance with at least one command transmitted through the power and communications bus, wherein the duration of the data pulse indicates a programming selection for the satellite and the at least one command further comprises a second data pulse, wherein a duration and a voltage level of the at least one data pulse directs the at least one programmable lead to act as an anode.

20-22. (canceled)
